(19) United States
(12) Patent Application Publication
(10) Pub. No.: US 2011/0256530 A1
(43) Pub. Date: Oct. 20, 2011

(54) PRODUCTS AND METHODS FOR TISSUE PRESERVATION

(75) Inventor: Michael Hogan, Tucson, AZ (US)

(73) Assignee: GenVault Corporation, Carlsbad, CA (US)

(21) Appl. No.: 13/085,302

(22) Filed: Apr. 12, 2011

Related U.S. Application Data
(60) Provisional application No. 61/323,185, filed on Apr. 12, 2010.

(51) Int. Cl. C12Q 1/68; C07H 21/00; G01N 1/28

(52) U.S. Cl. 435/6.1; 435/40.5; 536/23.1

(57) ABSTRACT

The present application relates to methods for increasing stability of formalin fixed, optionally paraffin embedded biological material. In one example, DNAse inhibitors and/or RNAse inhibitors are combined with the biological material or formalin at the time of fixation or shortly prior.

### CROSS-REFERENCE

This application claims the benefit of U.S. Provisional Application No. 61/323,185, filed Apr. 12, 2010, which application is incorporated herein by reference.

### FIELD OF THE INVENTION

This invention relates generally to stabilizing macromolecules or preserving tissue integrity, for example, during processing of the tissue for pathological analyses or for tissue research applications. The invention provides a set of small molecules or solutes for tissue preservation.

### BACKGROUND OF THE INVENTION

Aqueous formaldehyde (formalin) tissue fixation is the current gold-standard of medical pathology. During the early 20^{th} century, formalin fixation became highly optimized to support the use of dye and metallic stains. In the mid 20^{th} century, the same formalin fixation techniques were found to support the use of antibodies in immunohistochemistry, due to the stability and the small size of most protein epitopes.

The standard of solid tissue fixation in pathology (FFPE) is 10% phosphate buffered formalin, incubated at 25° C. for 8-24 hrs followed by dehydration in ethanol, solvent exchange into xylene, then embedding with a paraffin polymer blend having a melting temperature (Tₘ) of about 56° C. This process gives highly reproducible dye staining, but produces a DNA and RNA complement that has been fragmented, and modified internally with chemical damage.

DNA and RNA are not stable over long storage periods in fully dehydrated, paraffin embedded tissue. The data are reminiscent of what is seen for all nucleic acids when stored dry at ambient temperature in matrices such as filter paper. Such time dependent nucleic acid damage could result from (slow) hydrolysis resulting from residual water contamination, or even more likely, from the slow oxidation of nucleic acid bases (especially G) due to the diffusional interaction of the nucleic acid with molecular oxygen which had permeated into the FFPE tissue block.

The collateral nucleic acid damage incurred during aqueous formaldehyde processing greatly limits the applied genomics, due to nucleic acid instability and the large nucleic acid target size required for genomic testing—typically at least 500 bases. It has been shown that highly stabilized DNA and RNA can be obtained if formalin fixation is completely replaced, via the use of organic solvent mixtures. However, these “non-formalin” approaches have not been embraced by the pathology community because they produce small but very significant changes in tissue morphology, and because the solvent-based fixatives require significant change in a century of laboratory standard operating procedures.

### SUMMARY OF THE INVENTION

The present invention provides for the use of degradation inhibitors in the process of sample fixation to increase/maintain integrity of sample contents (e.g., sample nucleic acids, proteins, etc.). For example, in one embodiment, the compositions, methods, and kits herein can be used to inhibit loss or integrity of post-translational phosphate modification of protein (i.e., phosphor-proteins). In another embodiment, the compositions, methods, and kits herein can be used to inhibit degradation or maintain integrity of polynucleotides, purified or unpurified, including for example, DNA (dsDNA and ssDNA), methylated DNA, mitochondrial DNA, chloroplast DNA, DNA-RNA hybrids, RNA, mRNA, rRNA, or mixtures thereof, genes, chromosomes, plasmids, genomes of biological material such as microorganisms, e.g., bacteria, yeasts, viruses, viroids, molds, fungi, plants, animals, humans, and fragments thereof. Thus the degradation inhibitors can be polynucleotide degradation inhibitors or protein degradation inhibitors.

The sample herein can be further analyzed for in vitro diagnostic applications or research applications. In some instances, the samples herein are analyzed using nucleic acid analysis.

Examples of nucleic acid analysis that can be performed on sample nucleic acids after fixation according to the methods herein include but are not limited to: amplification such as single strand amplification, exponential amplification, PCR, digital PCR, quantitative PCR, real time PCR, sequencing including pyrosequencing, single strand extension sequencing, single molecule sequencing, sequencing by ligation, sequencing by hybridization, RFLP, SNP analysis, microarray analysis, etc.

The samples fixed herein can be further analyzed to detect or diagnose conditions including but not limited to: cancer, fetal abnormalities, autoimmune disorders, and other genetic conditions.

Examples of nucleic acid degradation inhibitors include, for example, nuclease inhibitors. Preferably, a nucleic acid degradation inhibitor used in the present invention is a small molecule, with the ability to permeate through a cell or tissue. A nucleic acid degradation inhibitor preferably inhibits polynucleotide degradation by at least 50%, 60%, 70%, 80%, 90%, 95%, 99%, or 99.5% when compared to degradation in the absence of the inhibitor. Concentrations of inhibitors used can vary. In some instances, the inhibitor(s) herein can added to an individual or final collective concentration of <0.05, 0.1 mM, 0.5 mM, 1 mM, 5 mM or 10 mM.

In some instances, a nucleic acid degradation inhibitor is a DNase inhibitor or an RNase inhibitor.

In some instances, multiple tissue-permeable, broad-spectrum nuclease inhibitors are added to a fixative (e.g., formalin) during the process of “fixation”. Addition of the broad-spectrum nuclease inhibitors in the fixative step results in greater integrity of DNA and/or RNA. For example, DNA may retain a length greater than about 1.0, 5.0, or 10 kb for over a week, month, year, or decade using the methods herein. Similarly, RNA may retain a length greater than about 0.1, 0.5, 1.0 or 10 kb for over a week, month, year, or decade using the methods herein.

Exemplary nuclease inhibitors are described herein in Table 1.

Exemplary ROS scavengers are described in Table 2.

In some embodiments, the degradation inhibitor is an inhibitor of the phosphoryl transferase superfamily.

In some embodiments, the degradation inhibitor is a small molecule. However, it can also be an enzyme or an antibody or other compound. Preferably the inhibitor has high tissue permeability. For example, an inhibitor of the invention can penetrate a tissue at a rate of >1 mm/hr, 5 mm/hr, 10 mm/hr, 20 mm/hr, 100 mm/hr. In some instances, degradation inhibitor of the invention penetrates tissue at a rate such that 50% of an inhibitor has penetrated the tissue within 4 hrs, 3 hrs, 2 hrs, 1 hr, 30 minutes, 20 minutes, 10 minutes, or 1 minute.

In some instances, a degradation inhibitor comprises a chelator. A chelator can be a Mg²⁺ chelator, or more preferably EDTA. In some aspects the chelator is used in combination with a RNase inhibitor or DNase inhibitor.

Preferably, a degradation inhibitor is small enough to readily diffuse into a pore that is less than 40 nm in diameter.

Degradation inhibitor can be employed singly or in combination. In some instances, at least 2, 3, 4, 5, 6, 7, 8, 9, or 10 different nucleic acid degradation inhibitors are added in the fixation step e.g., by addition to a fixative or an alcohol wash solution of ethanol. For example, combinations of nuclease inhibitors, ROS scavengers, and/or inhibitors of phosphoryl transferase superfamily can be added together.

As provided above, the inhibitor(s) can be added to the fixative. Or in alternative or in addition, they may be added to the alcohol wash (e.g., the first water-ethanol wash employed during tissue processing).

Thus, in some instances, the present invention relates to a composition comprising: at least 2, 3, 4, 5, 6, 7, 8, 9, 10 different degradation inhibitors, either all in a single container or in different containers but in a single kit. In some instances, the present invention contemplates a container comprising a fixative (e.g., formalin) and at least 1, 2, 3, 4, 5, 6, 7, 8, 9, 10 different degradation inhibitors within the same solution or container or in different solutions or containers but in the same kit. In yet further embodiments, the present invention contemplates a container comprising an alcohol wash (e.g., ethanol wash) and at least 1, 2, 3, 4, 5, 6, 7, 8, 9, 10 different degradation inhibitors within the same container or in different containers but in the same kit. Thus, in one embedment, the present invention relates to a solution comprising formalin and a degradation inhibitor. Preferably the degradation inhibitor is isolated or synthetic. Preferably, the degradation inhibitor is a small molecule. The solution can further comprise a sample. In some embodiments, the solution consists essentially of or consists of the formalin or other fixative and a degradation inhibitor.

The degradation inhibitors preferably retain intact DNA strand length and intact RNA strand length in formalin-fixed tissue after a month at 37° C., at values equal to or within 90%, 80%, 70%, 60%, 50%, 40%, 30%, 20%, 10%, 5%, 3%, or 1% difference from those obtained immediately upon completion of fixation.

The nucleic acid degradation inhibitors may be added before, or during the formalin fixation process.

Thus, the invention provides for a composition comprising, consisting, or consisting essentially of (i) one or more degradation inhibitors and (ii) formalin or alcohol wash solution.

The formalin, in any of the embodiments herein, may be phosphate buffered at between 1-37% formalin by mass.

Any of the compositions herein may further comprise a biological sample. The biological sample is preferably a tissue sample. The sample is preferably from a mammal or a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

As used herein the term “sample” or “biological sample” refers to a tissue sample, an organ sample, individual cell(s) or portions of any of the above. Preferred examples of samples include tissue samples, such as tissue biopsies for detection of cancer mutations (e.g., EGFR or HER2 mutations associated with unique prognosis or treatment selection) or aberrant expression of cancer genes. Other examples of samples include cheek swabs with intrabuccal cells, buccal cells, hair samples, blood samples, and mucus samples.

As used herein the terms “inhibition” and “inhibitors” also mean reduction in activity or compounds that reduce activity, e.g., wherein reduction in at least 50%, 40%, 30%, 20% of activity.

### BACKGROUND

The present invention provides reagents, methods, and kits for stabilizing macromolecules during preservation, for example, during processing of the tissue for pathological analyses or for tissue research applications. Specifically, the present invention relates to the addition of degradation inhibitors preferably nuclease inhibitors, to a formalin solution or to a sample before contacting with formalin solution to increase integrity of biomolecules in the sample (e.g., DNA, RNA, or proteins).

Formaldehyde is a gas, which upon saturation in water is hydrated to form methylene glycol (also known as “formalin”) which is the active agent during tissue permeation. That hydration is slowly reversible, and the resulting free COH₂, available at equilibrium, is responsible for the observed chemical modification of proteins and of nucleic acids. The desired chemical reaction between hydrated COH₂ and tissue is the crosslinking of very closely spaced amine groups on protein, to form the so-called formaldehyde crosslink or (—R₁—NH—CH₂—NH—R₂—) where, for instance, R₁ & R₂ might be closely spaced lysine or asparagine side-chains on adjacent proteins, or within the same protein. Such crosslinks occur very rapidly, if both reactants are within 0.1-0.2 nm, on the time average, and are associated with a relatively small, positive enthalpy of formation (i.e. process proceeds rapidly even in the cold). Those rapidly-forming formaldehyde crosslinks are the chemical linkages that provide the desired stabilization of tissue morphology for microscopy. As expected, the resulting bis-alkyl-amino crosslink is very stable with respect to hydrolysis in acid or base. In the absence of amine group proximity, the reaction stops at formation of the mono-amino adduct (—R₁—NH—CH₂—OH) which also forms readily in the cold, but is readily reversible in acid and base. Without being bound by theory some embodiments of the invention prevent or limit the degradation of polynucleotides while have no, or a limited effect, on the crosslinking.

The corresponding reaction between formalin and nucleic acid is a purely tissue side reaction and is undesirable for tissue preservation. The stable reaction products occurs with the exocyclic base amines, with the rough order of reactivity being A>C>G. As for protein-protein crosslinks, the only stable complexes to be formed are (—R₁—NH—CH₂—NH—R₂—) where most-often R₁ is the exocyclic amine and R₂ is a chromosomal protein (for DNA) or a RNP (for RNA). In the absence of such a bi-functional linkage, the monofunctional (—R₁—NH—CH₂—OH) adduct is readily reversible near neutrality, especially upon heating to about 70° C. Neither the monofunctional or crosslinking products induce DNA or RNA strand breaks, in simple nucleic acid solutions. That observation has been confirmed by Masuda et al. for RNA and by Tokuda et al. for DNA in a rat liver model in the field of chromatin immunoprecipitation (Chip) technology. Thus, the nucleic acid fragmentation observed during formalin fixation of solid tissues is not due to formaldehyde chemistry. Instead, it is a result of endogenous tissue nuclease activity during several hours of tissue soaking in a phosphate buffer. Accordingly, without being bound by theory, embodiments of the invention are directed at preventing the endogenous tissue nuclease activity during several hours of tissue soaking in a phosphate buffer.

Degradation Inhibitors and their Uses

The present invention relates to methods for reducing polynucleotide degradation and/or protein degradation during sample fixation. While fixation generally occurs by formalin, the present invention contemplates the use of degradation inhibitors with any fixative. Examples of non-formalin fixative include, but are not limited to, aldehydes, mercurials, alcohols, oxidizing agents, picrates, and an alcohol fixative. Without being bound by theory, the endogenous tissue nuclease activity may be due to the presence of water in the sample. In one embodiment, the invention further provides reagents and methods for elimination of water in the original fixation step in order to eliminate the endogenous tissue nuclease activity. For example, alcohol fixatives or fixation with alcohol-chloroform or alcohol-PEG may be use to eliminate water in the sample and to produce retention of a high molecular weight tissue complement.

Whichever fixative is used, the present invention contemplates the addition of one or more degradation inhibitors to the sample and/or fixative. For example, the degradation inhibitors can be polynucleotide degradation inhibitors and/or protein degradation inhibitors. Preferably, the fixative is formalin and the degradation inhibitor is not formalin. The degradation inhibitors can be isolated, naturally occurring agents or non-naturally occurring.

Degradation inhibitors can include any compound which reduces or inhibits degradation or fragmentation of nucleic acids or proteins. Thus, the terms “inhibition” or “inhibitor” or “inhibits” are synonymous with “reduction”, “reducer” or “reduces”, respectively. Reduction, as it pertains to degradation, is preferably at least 50%, 60%, 70%, 80%, 90%, 95% or 99% as compared to degradation in absence of an inhibitor for a set period of time. However, any reduction in degradation or integrity of nucleic acid will be considered an inhibition of degradation.

The degradation inhibitors include, but are not limited to inhibitors of the phosphoryl transferase superfamily such as chelators, DNAse inhibitors, RNAse inhibitors, and also scavengers or inhibitors of reactive oxygen species (ROS). In some cases, a degradation inhibitor is an enzyme. In some cases a degradation inhibitor is a small molecule. Preferably, it is water soluble. Preferably, it is poorly soluble in alcohol or xylene, so that upon tissue transfer to a water-free solvent, it is permanently embedded in the dehydrated tissue block matrix.

Chelators can be used as inhibitors of the phosphoryl transferase superfamily. Non-limiting examples of chelators included: (2-Hydroxypropyl)-beta-cyclodextrin solution; 2,3-Dimercapto-1-propanesulfonic acid sodium salt monohydrate; 3-Hydroxy-2-(5-hydroxypentyl)chromen-4-one; (+)-(18-Crown-6)-2,3,11,12-tetracarboxylic Acid; Aminocaproic Nitrilotriacetic Acid; α-Cyclodextrin; Aminocaproic Nitrilotriacetic Acid Tri-tert-butylester; Ammonium tartrate dibasic; BAPTA-tetramethyl Ester; BAPTA, Free Acid; Deferoxamine Mesylate; Dimethylglyoxime; DMSA (Meso-2,3-dimercaptosuccinic acid); EDTA, Disodium Salt, Dihydrate; EGTA; EGTA/AM; Ethylenediaminetetra(methylenephosphonic acid); sc-300682; Ethylenediaminetetraacetic acid diammonium salt; Ethylenediaminetetraacetic acid disodium salt solution; FLUO 3, Pentaammonium Salt; HBED; Heptakis(6-O-t-butyldimethylsilyl-2,3-di-O-acetyl)-β-cyclodextrin; INDO 1 pentapotassium salt; Iron DOTA Sodium Salt; MAPTAM; N-(2,6-Diisopropylphenylcarbamoylmethyl)iminodiacetic Acid; N,N-Dimethyldecylamine N-oxide; N,N-Dimethyldodecylamine N-oxide; N4,Nα,Nα,Nε,Nε-[Pentakis(carboxymethyl)]-N4-(carboxymethyl)-2,6-diamino-4-azahexanoic Hydrazide; Nitrilotriacetic acid; Nitrilotripropionic acid; Phenyleneethylenetriamine Pentaacetic Acid; Phytic acid hexabarium salt; Pyridoxal Isonicotinoyl Hydrazone; Potassium citrate monobasic; Potassium D-tartrate monobasic; Potassium oxalate monohydrate; Potassium sodium tartrate tetrahydrate; Potassium tetraoxalate dihydrate; rac (Bromoacetamidophenylmethyl)ethylenediaminetetraacetic Acid; RHOD 2 triammonium salt; RHOD 2/AM; [S-Methanethiosulfonylcysteaminyl]ethylenediamine-N,N,N′,N′-Tetraacetic Acid; (S)-1-(p-Bromoacetamidobenzyl)ethylenediaminetetraacetic Acid; Sodium bitartrate monohydrate; Sodium tartrate dibasic solution; sodium citrate, t-Boc-aminocaproicnitrilotriacetic Acid; Tetraacetoxymethyl Bis(2-aminoethyl) Ether N,N,N′,N′-Tetraacetic Acid; X-206; Zinpyr-1; or Zinpyr-4. Without being bound by theory it is thought that divalent cation chelaors, specifically M^{g2+} chelators, for instance EDTA, are effective at inhibiting DNAse, because of DNAse requires free M^{g2+} to function.

Examples of RNase inhibitors include but are not limited to Ribonuclease inhibitor (RI). RI is a large (˜450 residues, ˜49 kDa), acidic (pI˜4.7), leucine-rich repeat protein that forms extremely tight complexes with certain ribonucleases. RI is sensitive to oxidation. In some embodiments ROS scavengers are used to protect RI from oxidation. In such embodiments RI and a ROS scavenger are used in a common solution. There are several commercially available versions of RI which can be used in the methods, compositions and kits of the present invention. A non-exhaustive list of these includes: SUPERase·In™, RNAsecure™, Ambion® RNAlater®, RNAlater®-ICE, ribonucleoside-vanadyl complex, RNasin® Plus RNase Inhibitor, and RNAlater®. Oxidation-resistant ribonuclease inhibitors (described in U.S. Pat. No. 7,650,248) are another example of RNase inhibitors which can be used in the present invention.

Examples of DNAse inhibitors include but are not limited to inhibitors of deoxyribonuclease I, deoxyribonuclease II, and Micococcal nuclease. Non-limiting examples of DNAses include: N 2-mercaptoethanol; 2-nitro-5-thiocyanobenzoic acid; Actin; Alfatoxin B2a, G2, G2a, and M1 (non-competitive); Ca²⁺; EGTA and EDTA; Sodium dodecyl sulfate; Calf spleen inhibitor protein; and Carbodiimide and cholesterol sulfate. Other nuclease inhibitors include the compounds disclosed in US 2005/0214839, which is incorporated herein by reference.

Several phosphate antagonists are summarized in Table I.

TABLE I
Broad Spectrum Nuclease Inhibitors to be used in Solid Tissues

| | | | Relative | |
|---|---|---|---|---|
| | | | Diffusion | |
| | Molecular | Diameter | Coefficient | |
| Small Molecule | Weight | (nm) | (formalin) | Activity |
| Aluminum Fluoride AlF3 | 140 | 0.3 | 1.4 | Inhibitor of kinases and |
| | | | | nucleases |
| Sodium Ortho-vanadate Na3VO4 | 183 | 0.3 | 1.5 | Inhibitor of kinases and |
| | | | | nucleases |
| EDTA | 292 | 0.4 | 1.7 | Divalent Chelator, Inhibitor |
| | | | | of DNases& some kinases |
| Uridine-Vanadate U-V04 | 350 | 1.0 | 1.9 | RNase specific Inhibitor |
| Aurinetricarboxylic Acid (ATA)n | (422) × n = | 1.0 × 2.0 | 3.5 (for n = | Triphenyl methane |
| | 2,100 (for n | (for n = 5) | 5) | derivative, broad spectrum |
| | = 5) | | | inhibitor of kinases & |
| | | | | nucleases |
| Porcine Ribonuclease Inhibitor | 47,000 | 15 | 10 | High affinity, specific to |
| | | | | RNase: too large to be |
| | | | | useful in tissues |

As noted in Table I, there are a number of broad spectrum, small molecule inhibitors of the phosphoryl transferase family, with a size and diffusion coefficient that are comparable to that of EDTA. Without being bound by theory solid tissue diffusional dynamics suggest that molecules as small as those in Table I should be able to diffuse into tissue spaces during the first 1-4 hours of tissue fixation. Preferably, such inhibitors added to the fixation step have a faster rate of inhibiting nucleases than formalin. In one example, a degradation inhibitor of the invention inhibits nucleases at a rate of at least 2, 3, 4, 5, 6, 7, 8, 9, or 10 than formalin alone when used alone under the same concentrations. Thus, in one embodiment, the degradation inhibitors comprise a set of generalized nuclease inhibitors with the ability to co-diffuse with formalin into tissues and, via concurrent inhibition of tissue nucleases, with the ability prohibit fragmentation of DNA and RNA during the earliest stages of formalin fixation.

Other examples of degradation inhibitors include ROS scavengers. Reactive oxygen species (ROS) are the chemical species which have a single unpaired electron in their outer shell configuration. ROS are chemically reactive and can damage polynucleotides by addition to one or more sites in the nucleic acid structure. Oxygen ions are an example or ROS. Oxygen damage is mediated via thermal conversion of ordinary (ground) triplet state O₂, to form the excited singlet state O₂ radical and subsequent peroxide and hydrated electron equivalents which can form upon electron exchange with residual water in the matrix. This mixed class of excited-state O₂ is referred to as the reactive oxygen species (ROS).

Degradation inhibitors can comprise ROS scavengers. Non-limiting examples of ROS scavengers include: Tiron, SOD (Super oxide dismutase), Catalase, Glutathione-peroxidase, Glutathione-reductase, Super-oxide reductases, Vitamin-E, Vitamin-C, Flavonoids, Vitamin-B, Carotenoids, Lipoic acid, Copper, Zinc, and Selinium.

A number of well-studied water soluble, small-molecule ROS scavengers with a range of useful chemical properties are listed in Table II.

TABLE II
Reactive Oxygen Species Scavengers

| | | | Relative | |
|---|---|---|---|---|
| | | | Diffusion | |
| | | | Coefficient | |
| | Mol | Diameter | (vs | Useful Chemical |
| ROS Scavenger | Wt | (nm) | formalin) | Properties |
| Histidine | 155 | 0.3 | 1.4 | Neutral zwitterions |
| | | | | at pH 7 (30) |
| Mannitol | 182 | 0.3 | 1.5 | Neutral (31) |
| Anserine (Alanine- | 240 | 0.4 | 1.6 | Cation at pH 7 (32) |
| methyl-histidine) | | | | |
| Ascorbate | 176 | 0.3 | 1.5 | Anion at pH 7 (33) |

In one embodiment, the invention provides water-soluble ROS scavengers in a first 50% ethanol-water alcohol wash, immediately subsequent to formalin fixation. In another embodiment, the water-soluble ROS are small molecules that can readily diffuse into the porous tissue spaces along with water and ethanol. In various embodiments the water-soluble ROS scavengers can be included in a water wash. In various embodiments the water-soluble ROS scavengers can be included in a less than 10%, less than 20%, less than 30%, less than 40%, less than 50%, less than 60%, less than 70%, less than 80%, less than 90%, or less than 95% alcohol wash. Without being bound by theory it is believe that after introduction of the ROS scavenger into the tissue block, an ethanol-water soaking step, it would subsequently be trapped in the tissue during the subsequent ethanol-rich wash steps, thus permanently entrapping the scavenger in the tissue block, to quench ROS as they are created during long term tissue block storage.

In other embodiments ROS scavengers can be added to one or more solutions during fixation and preservation of a tissue sample. The ROS scavengers can be added to a buffered solution, to formalin, to alcohol washes, or to some or all of these solutions. The sample is then transferred through these solutions which can contain the ROS scavengers.

The degradation inhibitors are generally small enough to diffuse into tissue. The diffusion can occur along with formalin or during an ethanol wash. The diffusion can also occur while the sample is in paraffin. In particular embodiments the degradation inhibitors can diffuse readily through a pore that is less than 50 nm, 40 nm, 30 nm or 20 nm. Thus, a degradation inhibitor is preferably smaller than 50 nm, 40 nm, 30 nm or 20 nm in diameter.

The degradation inhibitors can be used individually or in combination (i.e., at least 1, 2, 3, 4, 5, 6, 7, 8, 9 or 10 different inhibitors) by contacting them with a fixative such as formalin or with a biological sample prior to fixation by the fixative. For example, an RNase inhibitor can be used in conjunction with a chelator such as EDTA. The two inhibitors can be added to formalin or other fixative prior to, simultaneous with, or after contacting the fixative with the biological sample.

In certain embodiments, the degradation inhibitors inhibit both DNase and RNase.

In some embodiments, the degradation inhibitor(s) are in a solution with only formalin and phosphate buffer. In some embodiments a solution of the invention consists essentially of degradation inhibitor, formalin and phosphate buffer. In some embodiments a solution of the invention consists essentially of degradation inhibitor, water, and an alcohol. In some embodiments a solution of the invention consists of degradation inhibitor, water, and an alcohol.

In some embodiments, the degradation inhibitor(s) herein is a water-soluble reactive oxygen species scavenger, a water-soluble reactive oxygen species scavenger, or a reactive oxygen species selected from the compounds disclosed in Table 2.

In some embodiments, the compositions herein further comprise a chelator.

In some embodiments, the degradation inhibitor is a combination of one or more small molecule inhibitors of the phosphoryl transferase superfamily and one or more water-soluble reactive oxygen species scavengers. In some embodiments the degradation inhibitors are chosen from the compounds listed in Table 1 and Table 2.

As described above, the degradation inhibitors of the invention can be used in preservation and/or storage of a polynucleotide from a biologic sample. Such method comprises: contacting the biologic sample with formalin, and contacting the biologic sample with a degradation inhibitor. The formalin pH can be in the range of 3 to 10, or 6 to 8. The degradation inhibitors can be stored in a buffered solution or the formalin can be buffered. In one instance the buffered solution is a phosphate buffer solution. The degradation inhibitors, especially the non-water soluble ones such as the non-water soluble ROS inhibitors, may in some embodiments be added to paraffin. In some embodiments, the degradation inhibitors are in multiple solutions, for instance the degradation inhibitors is in a buffered solution with formalin and in a series of ethanol washes for dehydration. A tissue sample is them moved sequentially through these solutions. In some embodiments the temperature is controlled in some or all of the solutions used during the method. In some embodiments, the contacting the biologic sample with formalin is performed at a temperature between 1° C. and 6° C.

In some embodiments, the invention provides for methods for analyzing a polynucleotide comprising: fixing said polynucleotide with a degradation inhibitor and a fixing agent (optionally embedding, and storing the fixed polynucleotide), then purifying the polynucleotide, and analyzing it.

In some instances, a fixed and embedded polynucleotide is purified using any of the Examples provided below.

Analysis of recovered polynucleotides can include any of the following steps: amplification such as single strand amplification, exponential amplification, PCR, digital PCR, quantitative PCR, real time PCR, sequencing including pyrosequencing, single strand extension sequencing, single molecule sequencing, sequencing by ligation, sequencing by hybridization, RFLP, SNP analysis, FISH, mass spectrometry, radiolabelling, RNA expression analysis, whole transcriptome analysis, etc.

Data from analysis can be used for to detect or diagnose a condition including but not limited to: cancer, fetal abnormalities, autoimmune disorders, and other genetic conditions. Data analysis can be transmitted over the internet or via wireless connections from one computer terminal to another (including cell phones). By increasing stability of DNA and RNA (for example) samples can be shipped off-shore to foreign countries to be analyzed and the analysis/diagnosis can be transmitted back to this country.

The step of contacting the biologic sample with a degradation inhibitor can be performed while the degradation inhibitor is in a fixative solution, an alcohol wash, or both.

In some instances, the inhibitor(s) herein can added such that their individual or collective final concentration within the fixative solution is <0.05, 0.1 mM, 0.5 mM, 1 mM, 5 mM or 10 mM. In other instances, their individual or collective final concentration within the fixative solution is at least 0.05, 0.1 mM, 0.5 mM, 1 mM, 5 mM or 10 mM.

In some embodiments, the invention discloses formalin fixed optionally paraffin embedded biological sample or tissue containing a plurality of polynucleotides wherein at least 5% of the polynucleotide strands in the sample are greater than 100, 200, 300, 400, or 500 base pairs. In some embodiments the invention discloses a formalin fixed paraffin embedded tissue containing a plurality of polynucleotide strands wherein at least 1% of the polynucleotide strands in the sample are greater than 100, 200, 300, 400, or 500 base pairs. In some embodiments the invention discloses a formalin fixed paraffin embedded tissue containing a plurality of polynucleotides wherein at least 1%, 5%, 10%, 20%, 30%, 40%, 50%, 60%, 70%, 80% or 90% of the polynucleotide strands in the sample are greater than 100, 200, 300, 400, or 500 base pairs.

In some embodiments, the invention discloses a formalin fixed paraffin embedded tissue containing a plurality of deoxynucleotides wherein at least 5% of the deoxynucleotide strands are greater than 500, 100, 200, 300, 400 or 500, or 1000 base pairs. In some embodiments, at least 1%, 5%, 10%, 20%, 30%, 40%, 50%, 60%, 70%, 80% or 90% of the deoxynucleotide strands from a sample are greater than 500, 100, 200, 300, 400 or 500, 1000, 5000, or 10,000 base pairs. In some embodiments at least 10% of the deoxynucleotide strands from a sample are greater than 100, 200, 300, 400, 500, 600, 700, 800, 900, 1,000, or 10,000 base pairs.

In some embodiments, the invention discloses a formalin fixed paraffin embedded tissue containing a plurality of ribonucleotides wherein at least 5% of the ribonucleotides are greater than 500, 100, 200, 300, 400 or 500, or 1000 base pairs. In some embodiments at least 1%, 5%, 10%, 20%, 30%, 40%, 50%, 60%, 70%, 80% or 90% of the ribonucleotides are greater than 500, 100, 200, 300, 400 or 500, or 1000 base pairs. In some embodiments at least 10% of the ribonucleotides are greater than 1000 base pairs.

In some embodiments the a formalin fixed paraffin embedded tissue containing a plurality of non-degraded polynucleotides is stored for more than 1, 2, 3, 4, 5, 6, 7, 8, 9, 10, 11, or 12 months. In some embodiments the a formalin fixed paraffin embedded tissue containing a plurality of non-degraded polynucleotides is stored for more than 1, 2, 3, 4, 5, 6, 7, 8, 9, 10, 11, or 12 years. In some embodiments, formalin fixed paraffin embedded tissue treated a with a degradation inhibitor has more than 10%, 20%, 30%, 40%, 50%, 60%, 70%, 80% or 90% less degradation of a polynucleotide than a formalin fixed paraffin embedded tissue not treated with a degradation inhibitor. The comparison between the samples can occur more than 1, 2, 3, 4, 5, 6, 7, 8, 9, 10, 11, or 12 months after fixation. The comparison between the samples can occur more than 1, 2, 3, 4, 5, 6, 7, 8, 9, 10, 11, or 12 years after fixation. Thus, the present invention contemplates a method for reducing effective degradation of polynucleotides in a sample to be fixed comprising: contacting said sample with a fixative, and contacting said sample with one or more polynucleotide degration inhibitors. The fixed sample can then be optionally embedded, e.g., with paraffin.

In some embodiments, the invention provides for a polynucleotide recovered from fixed sample wherein (i) optionally, polynucleotide is at least 500 base pairs or at least 50%, 60%, 70%, 80%, 90% or 95% of the polynucleotides in the sample are at least 500 base pairs long, (ii) the sample was fixed in a fixative comprising one or more degradation inhibitors, and (iii) optionally, the fixed sample was embedded in paraffin. Alternatively, a polynucleotide of the invention may be recovered from fixed sample wherein (i) optionally, the polynucleotide is at least 500 base pairs or at least 50%, 60%, 70%, 80%, 90% or 95% of the polynucleotides in the sample are at least 500 base pairs long, (ii) the sample was fixed in a fixative (e.g., formalin), (iii) the sample was then washed in an alcohol wash such as an ethanol-water wash comprising one or more degradation inhibitors, and (iv) optionally, the fixed sample was embedded in paraffin.

In some embodiments of the invention the formalin has a pH in the range of 3-10, 3-4, 4-5, 5-6, 6-7, 7-8, 8-9, or 9-10.

In some embodiments, the degradation inhibitor is a small molecule. However, it can also be an enzyme or an antibody or other compound. Preferably the inhibitor has high tissue permeability. For example, an inhibitor of the invention can penetrate a tissue at a rate of >1 mm/hr, 5 mm/hr, 10 mm/hr, 20 mm/hr, 100 mm/hr In some instances, degradation inhibitor of the invention penetrates tissue at a rate such that 50% of an inhibitor has penetrated the tissue within 4 hrs, 3 hrs, 2 hrs, 1 hr, 30 minutes, 20 minutes, 10 minutes, or 1 minute.

### Temperature

In some embodiments, the method provides optimization for formalin tissue fixation at a temperature within the range of about 4° C. (in an ice bucket) to about 25° C. (room ambient). Without being bound by theory it is thought that for duplex DNA, the energetics for formaldehyde adduct formation are highly unfavorable because the exocyclic base plane amines (the site of formaldehyde adduct formation) are only available for chemical reaction after transient thermal disruption of the double helix (i.e. helix “breathing”). Similarly, for partially structured RNA strands, or for RNA strands that are obscured by complexation with a ribonucleoprotein, the intramolecular or intermolecular RNA complexes will also tend to occlude interaction with solvated formaldehyde and will be made more available upon their thermal disruption. Thus, the chemical crosslinking of closely-spaced structural proteins in the tissue lattice (the desired reaction with formaldehyde) will, especially at low temperature, be much faster than (undesired) nucleic acid adduct formation in the same tissue. At 25° C., formalin will permeate through about 1 mm of tissue block per hour: thus specifying that a 1 cm tissue block would take at least 12 hrs. During most of that incubation period, the tissue is not crosslinked, and thus, it is expected that DNase and RNase activity in those tissues would persist exactly as if the tissue block were kept at room temperature (RT) for 5 hrs in ordinary phosphate buffered saline. It has been shown that nearly complete degradation of rRNA occurs during such an unprotected 12-hour, room temperature “preanalytic” tissue incubation at 25° C.

In some embodiments the temperature is controlled while a sample is exposed to degradation inhibitors. In some embodiments the temperature is between 1° C. and 6° C. while the sample is exposed to degradation inhibitors. In some embodiments the temperature is controlled in steps which occur prior to contacting the sample with degradation inhibitors. For instance a sample may be fixed in 1° C. to 6° C. formalin and then exposed to degradation inhibitors in subsequent alcohol washes.

In some embodiments of the invention the formalin contacts the biologic sample at a temperature less than 25° C., between 0° C. and 6° C., chilled on ice prior to contacting the sample, or between 1° C. and 3° C. An alcohol solution that contacts the biologic sample can be at a temperature less than 25° C., between 0° C. and 6° C., chilled on ice prior to contacting the sample, or between 1° C. and 3° C. Preferably, the fixation occurs in room temperature. In some instances, fixation occurs at 4-40° C., 10-30° C. or about 25° C.

### Low Oxygen Environment

Another way to reduce damage to a polynucleotide during storage is to limit the amount of oxygen present during storage of a sample. Accordingly, some embodiments of the invention are directed at using the compounds of the invention or performing the methods of the invention in a reduced oxygen environment. In one embodiment, storage is performed in a low oxygen, high nitrogen environment. For instance a multiple gas control system using an O₂ system with a high-precision oxygen sensor can be used to regulate oxygen and add nitrogen to an incubator. In some embodiments the oxygen level is less than 15%, less than 10%, less than 5%, or less than 1%.

### Reduced Mg²⁺Reagents.

In some embodiments reagents for use with the compounds of the invention or for use in the methods or kits of the invention use water that has been treated to reduce the Mg^{2+.}For example distilled water, or “softened water” made with an ion exchange resin or by ion exchange chromatography is used.

### Kits

A kit of the invention can include one or more of the following: a container for storing a biological sample, one or more degradation inhibitors, a formalin solution, an ethanol solution, and a paraffin or other material to embed a sample. The degradation inhibitors can be a an inhibitor of the phosphoryl transferase superfamily, any compound described in Table 1, a reactive oxygen species scavenger, any compound described in Table 2, any other DNAse inhibitor or RNAse inhibitor. Preferably, the degradation inhibitors are water soluble. Preferably, the degradation inhibitors are small molecules.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Fixation of a Samples

Many samples will be fixed using different compounds and methods disclosed herein. These samples will be used to compare the different anti-polynucleotide degradation characteristics of the invention.

Fixation is the first step in any procedure in which tissue is to be preserved for histological study. Common fixatives will be used to kill the tissue, any bacteria present in the tissue, and to cross-link sample proteins. Common fixatives include: Buffered formalin, 4% formaldehyde in buffered isotonic saline, Bouin's fluid, Picric acid, and Carnoy's fixative. These fixatives will be used in combinations with agents described herein to prevent the degradation of polynucleotide's in the sample.

Dehydration, or the removal of water from the tissue and replacement with ethanol, will occur next A graded series of mixtures of water and ethanol, generally from 50%-70% to 100% ethanol, will be used. This will also server to remove the fixative. In some instances distilled or softened water will be used and compared to water sources with more Mg²⁺. In some instances compounds disclosed herein will be added to the ethanol wash. For example ROS scavengers will be added in some instances.

Clearing, where the 100% ethanol is replaced by solvent miscible with the embedding medium, will be performed next. When using paraffin the solvent is usually xylene. As the tissues become infiltrated with xylene it will become more transparent (clearing). A first a mixture of 50% ethanol and 50% xylene followed by 100% xylene for an hour each will be used.

Infiltration will occur next. Infiltration is the process by which the xylene is replaced by paraffin. First a 50:50 mixture of xylene (30 minutes) and paraffin followed by two changes of 100% paraffin will occur. Infiltration typically occurs in an oven at 58-60° C.

Next the tissue will be oriented and embed in a paraffin block. The block will be placed in ice water to solidify.

### Example 2

Embedded tissues will be mounted on microscope slides:

The paraffin embedded tissue from Example will now be trimmed to a trapezoid shape and then placed in the chuck of a microtome. A microtome is mechanical device that advances the tissue a fixed amount (1-10 mm) as it moves the block of tissue up and down so that the block passes over a knife that cuts the paraffin and tissue into thin sections. When done correctly the successive (serial) sections form a ribbons.

The paraffin ribbons will then be transferred to a storage box or directly to microscope slides that has been coated with egg albumen with the aid of a small brush. The albumen acts as an adhesive and sticks the sections to the slide. Compounds of the invention can be added to the adhesive in order to prevent the degradation of polynucleotides on the slide.

The slides will then be placed on a warming tray and distilled water is added to float the paraffin sections and allow then to expand and straighten out. The excess water will be removed and the slides dry and sections will adhere to the slides.

### Example 3

### Extraction of DNA from Formalin-Fixed Paraffin Embedded Tissue Using GenElute™ Mammalian Genomic DNA Miniprep Kit (Product No. G1N10) from Sigma-Aldrich

A small section (about 20 mg) of paraffin-embedded tissue treated as claimed herein will be placed in a 2 ml microcentrifuge tube. 1200 μl of xylene will be added. The sample will be vortexed for 30 seconds. The sample will be centrifuged at full speed for 5 minutes at room temperature. Next the supernatant will be removed by pipetting without removing any of the pellet. 1200 μl of ethanol will be added to the pellet to remove the residual xylene. Next the sample will be mixed by vortexing and centrifuged at full speed for 5 minutes at room temperature. The ethanol will be carefully removed by pipetting without removing any of the pellet. An second ethanol wash will be performed. The open microcentrifuge tube will then be incubate at 37° C. for 10-15 minutes to remove any residual ethanol by evaporation. The tissue will be digested by resuspending the tissue pellet in 180 μl of Lysis Solution T. 20 μl of Proteinase K will be added and the solution will be mixed by vortexing. The solution will then be incubated at 55° C. overnight or until the tissue is completely lysed with occasional vortexing during incubation. The cells will be lyses by vortexing for 15 seconds, add 200 μl of Lysis Solution C to the sample, and vortexing thoroughly as a homogenous mixture is essential for efficient lysis. The solution will then be incubated at 70° C. for 10 minutes. 500 μl of the Column Preparation Solution will be added to pre-assembled GenElute™ Miniprep Binding Column and the samplw will be centrifuged at 12,000×g for 1 minute. 200 μl of ethanol will be added to the lysed sample and mixed by vortexing. The entire contents of the sample tube will be transferred into the treated binding column and centrifuge at ≧6500×g for 1 minute. The collection tube containing the flow-through liquid will be discarded and the binding column will be placed in a new 2 ml collection tube. Prior to first use, a wash will be performed. The Wash Solution Concentrate will be diluted with ethanol as. 500 μl of Wash Solution will be added to the binding column and centrifuged for 1 minute at ≧6,500×g., followed by discarding the collection tube containing flow-through liquid and placing the binding column in a new 2 ml collection tube. A second wash will then be performed. 500 μl of Wash Solution will be added to the binding column and centrifuged for 3 minutes at maximum speed (12,000-18,000×g) to dry the binding column, followed by discarding the collection tube containing flow-through liquid and placing the binding column in a new 2 ml collection tube. Next the DNA will be eluted by pipetting 200 μl of the Elution Solution directly into the center of the binding column, incubating at room temperature for 5 minutes, and centrofuginh for 1 minute at ≧6500×g to elute the DNA. The DNA samples will be stored at −20° C.

### Example 4

### Extraction of DNA from Formalin-Fixed Paraffin Embedded Tissue Using BiOstic® FFPE Tissue DNA Isolation Kit (Cat. No. 12250-50; MO BIO Laboratories)

Solution FP1 and FP2 will be added to the tube containing up to 15 mg of FFPE tissue samples which has been fixed according to the methods and compositions of the present invention. Solution FP3 will be added and heated at 55° C. for 1 h followed by 90° C. for 1 h. To separate the debris from the digested lysate and transfer to a clean tube the sample will be centrifuged. Solution FP4 and Solution FP5 will be added and mixed. The mixture will then be loaded onto a spin filter and centrifuged to bind the DNA to the silica membrane. The filter will be washed and spun with Solution FP6, the flow-through decanted and washed with Solution FP7. The flow-through will then be discarded and the filter centrifuged for 2 min to dry the membrane. The solution will then be transfer to the final elution tube and the purified DNA will be eluted in in Solution FP8. DNA will then be ready for use in qPCR or PCR applications.

### Example 5

### Extracting Total RNA from Formalin-Fixed, Paraffin-Embedded (FFPE) Tissue Samples for Use With the SOLiD™ SAGE™ Kit (Part No. 4452811) or SOLiD™ SAGE™ Kit with Barcoding Adaptor Module (Part No. 4443475). This Method Uses the RecoverAll™ Total Nucleic Acid Isolation Kit for FFPE (Part No. AM1975). All from Applied Biosystems™

The following RecoverAll™ procedure will be performed on samples fixed according to the methods and compositions of the present invention.

Treat with Xylene to remove paraffin:
1. Add 1 mL 100% xylene to each sample.
2. Vortex briefly to mix.
3. Centrifuge briefly to bring any tissue that is stuck to the sides of the tube down into the xylene.
4. Heat the sample for 3 minutes at 50° C. to melt the paraffin.
5. Centrifuge the sample for 2 minutes at room temperature and maximum speed to pellet the tissue.
6. Optional: If the sample does not form a tight pellet, recentrifuge for an additional 2 min. If a tight pellet still does not form, then proceed with caution in the following step.
7. Remove the xylene without disturbing the pellet. Discard the xylene according to applicable regulations.

Ethanol wash
8. Add 1 mL of 100% ethanol (room temperature) to the sample and vortex to mix. The tissue should turn opaque.
9. Centrifuge the sample for 2 minutes at room temperature and maximum speed to pellet tissue.
10. Remove and discard the ethanol without disturbing the pellet. The ethanol will contain trace amounts of xylene, and must be discarded accordingly.
11. Repeat steps 8-10 above to wash a second time with 1 mL of 100% ethanol.
12. Briefly centrifuge again to collect any remaining drops of ethanol. Remove as much residual ethanol as possible without disturbing the pellet.
13. Dry in a centrifugal vacuum concentrator at 40-45° C. for <20 minutes or 37-40° C. for 20-40 minutes. You may also air dry the pellet for 45 minutes at room temperature, though larger tissue sections may not dry completely.

Digest with Protease
14. Add 200 μL Digestion Buffer to each sample.
15. Add 4 μL Protease to each sample.
16. Swirl the tube gently to mix and immerse the tissue. If the tissue sticks to the sides of the tube, use a pipette tip to push it into the solution, or briefly centrifuge to bring the tissue down into the solution.
17. Incubate the sample in heat blocks for 15 minutes at 50° C., then 15 minutes at 80° C. to clarify the sample.
18. In a separate tube, combine 2.3 mL 100% ethanol with 1 mL Isolation Additive (enough for four samples, plus overage).
19. Add 790 μL of the Isolation Additive/ethanol mixture to each sample and pipette up and down.
20. For each sample, place a Filter Cartridge in one of the Collection Tubes supplied in the kit.
21. Pipet up to 700 μL of each sample mixture onto the Filter Cartridge and close the lid. Avoid pipetting large pieces of undigested tissue onto the Filter Cartridge.
22. Centrifuge at 10,000×g (typically 10,000 rpm) for 30 sec to pass the mixture through the filter.
23. Discard the flow-through, and re-insert the Filter Cartridge in the same Collection Tube.
24. Repeat steps 20-23 until all the sample mixture has passed through the filter.

Wash
25. Add 700 μL of Wash 1 to the Filter Cartridge.
26. Centrifuge for 30 sec at 10,000×g to pass the mixture through the filter.
27. Discard the flow-through, and re-insert the Filter Cartridge in the same Collection Tube.
28. Add 500 μL of Wash 2/3 to the Filter Cartridge.
29. Centrifuge for 30 sec at 10,000×g to pass the mixture through the filter.
30. Discard the flow-through, and re-insert the Filter Cartridge in the same Collection Tube.
31. Spin the assembly for an additional 30 sec to remove residual fluid from the filter.

Treat with DNase
32. In a separate tube, combine 27 μL 10× DNase Buffer, 18 μL DNase, and 225 μL nuclease-free water (enough for four samples, plus overage).
33. Add 60 μL of the DNase mix to the center of each Filter Cartridge.
34. Cap the tube and incubate for 30 minutes at room temp (22-25° C.).

Final wash
35. Add 700 μL of Wash 1 to the Filter Cartridge.
36. Incubate for 30-60 sec at room temperature.
37. Centrifuge for 30 sec at 10,000×g.
38. Discard the flow-through, and re-insert the Filter Cartridge in the same Collection Tube.
39. Add 500 μL of Wash 2/3 to the Filter Cartridge.
40. Centrifuge for 30 sec at 10,000×g.
41. Discard the flow-through, and re-insert the Filter Cartridge in the same Collection Tube.
42. Repeat steps 39-41 to wash a second time with 500 μL of Wash 2/3.
43. Centrifuge the assembly for 1 minute at 10,000×g to remove residual fluid from the filter.

Elute
44. Transfer the Filter Cartridge to a fresh Collection Tube.
45. Apply 60 μL of room-temperature Elution Solution or nuclease-free water to the center of the filter, and close the cap.
46. Allow the sample to sit at room temperature for 1 min.
47. Centrifuge for 1 minute at maximum speed to pass the mixture through the filter. The eluate contains the RNA.
48. Pool the elutions into a single tube. Store at −20° C. or colder. To store your sample for an extended period of time, transfer the eluate to a non-stick tube to prevent loss of nucleic acid.

1. A composition comprising:
a. a degradation inhibitor, and
b. formalin.

2. The composition of claim 1 wherein the formalin is phosphate buffered at between 1-37% by mass.

3. The composition of claim 1 further comprising a solid tissue sample.

4. (canceled)

5. The composition of claim 1 wherein the degradation inhibitor comprises a small molecule inhibitor of the phosphoryl transferase superfamily.

6.-12. (canceled)

13. The composition of claim 1 wherein the degradation inhibitor a reactive oxygen species scavenger.

14.-16. (canceled)

17. A composition comprising:
a. a small molecule inhibitor of the phosphoryl transferase superfamily, and
b. a reactive oxygen species scavenger.

18.-20. (canceled)

21. A method for preserving a polynucleotide in a biologic sample comprising:
a. contacting the biologic sample with formalin,
b. contacting the biologic sample with a degradation inhibitor.

22. The method of claim 21 wherein the contacting the biologic sample with formalin is performed at a temperature between 1° C. and 6° C. or at room temperature.

23. The method of claim 21 wherein the formalin has a pH in the range of 3 to 10.

24. The method of claim 21 wherein the degradation inhibitor comprises a small molecule inhibitor of the phosphoryl transferase superfamily.

25. The method of claim 24 wherein the small molecule inhibitor comprises one or more of (Table 1).

26. The method of claim 21 wherein the degradation inhibitor comprises a water-soluble reactive oxygen species scavenger.

27. The method of claim 26 wherein the water-soluble reactive oxygen species scavenger comprises one or more of (table 2).

28. The method of claim 21 wherein the degradation inhibitor comprises a small molecule inhibitor of the phosphoryl transferase superfamily and a water-soluble reactive oxygen species scavenger.

29. The method of claim 28 wherein the small molecule inhibitor comprises one or more of (Table 1) and wherein the water-soluble reactive oxygen species scavenger comprises one or more of (table 2).

30. A method for analyzing a polynucleotide comprising
a. obtaining a biologic sample, wherein the sample has been contacted with formalin solution, contacted with a degradation inhibitor, dehydrated, embedded, and stored;
b. purifying the polynucleotide, and
c. analyzing the polynucleotide.

31.-45. (canceled)

46. A formalin fixed paraffin embedded tissue containing a plurality of polynucleotides wherein at least 5% of the polynucleotides strands are greater than 300 base pairs.

47. A polynucleotide recovered from fixed tissue:
a. wherein the polynucleotide strand is at least 300 base pairs,
b. wherein the tissue was fixed in formalin,
c. wherein the formalin comprises a degradation inhibitor.
d. wherein the fixed tissue was embedded in paraffin.

48.-53. (canceled)

54. A polynucleotide recovered from fixed tissue:
a. wherein the polynucleotide strand is at least 300 base pairs,
b. wherein the tissue was fixed in formalin,
c. wherein the tissue was washed in a ethanol-water wash comprising a water-soluble reactive oxygen species scavenger, and
d. wherein the fixed tissue was embedded in paraffin.

55. The polynucleotide of claim 54 wherein the reactive oxygen species scavenger comprises one or more of (Table 2).
